(19) United States
(12) Patent Application Publication
(10) Pub. No.: US 2011/0301535 A1
(43) Pub. Date: Dec. 8, 2011

(54) MICROFLUIDIC CONTROL SYSTEMS

(75) Inventors: Shuichi Takayama, Ann Arbor, MI (US); Bobak Mosadegh, Irvine, CA (US)

(73) Assignee: THE REGENTS OF THE UNIVERSITY OF MICHIGAN, Ann Arbor, MI (US)

(21) Appl. No.: 13/119,359

(22) PCT Filed: Sep. 2, 2009
(86) PCT No.: PCT/US2009/055722

§ 371 Date: Jun. 17, 2011

Related U.S. Application Data
(60) Provisional application No. 61/104,492, filed on Oct. 10, 2008.

(51) Int. Cl. A61M 5/00; C12M 1/34; C12Q 1/02; B01L 3/00; G01N 33/566

(52) U.S. Cl. 604/93.01; 422/502; 435/7.92; 435/29; 435/287.1; 436/501

(57) ABSTRACT

The present invention relates to microfluidic devices. In particular, the present invention relates to microfluidic devices for performing spatio-temporal operations and applications thereof.

### CROSS REFERENCE TO RELATED APPLICATIONS

This application claims priority to U.S. Provisional Application Ser. No. 61/104,492, filed on Oct. 10, 2008, which is herein incorporated by reference in its entirety.

### GOVERNMENT SUPPORT

This invention was made with government support under Grant HL-084370, awarded by the National Institutes of Health. The government has certain rights in the invention.

### FIELD OF THE INVENTION

The present invention relates to microfluidic devices. In particular, the present invention relates to microfluidic devices for performing spatio-temporal operations and applications thereof.

### BACKGROUND OF THE INVENTION

Despite the advantages that microfluidics provides in terms of lower material consumption, faster reaction times, multiplexing, and ability to provide physiological cell culture microenvironments, widespread use of microfluidic circuitry in the lab and clinic are still limited. One of the challenges is that unlike electronic systems where the controller and actuator circuits are all electrical current driven, current microfluidics require electrical circuitry in addition to fluid flow for control and actuation when performing complex functions. This inevitably complicates overall device architecture by the need for integration, alignment, and interfacing of electrical components, actuators, and power sources with the microfluidics components. What would benefit practical system construction is the development of logic-embedded microfluidic circuitry where all of the system controllers as well as actuators are fluid flow driven (for example by using compressed gas). The difficulty to create such a system is that embedded logic requires integration of multiple non-linearly responding components whereas low Reynolds number microfluidic systems are typically linear in their response.

What is needed are simple systems that operate with minimal or no logical input.

### SUMMARY OF THE INVENTION

The present invention relates to microfluidic devices. In particular, the present invention relates to microfluidic devices for performing spatio-temporal operations and applications thereof.

In some embodiments, the present invention provides a system, comprising: one or more microfluidics devices, wherein each of the microfluidic devices comprises two or more segmented species-containing channels, where the pressure of the species joins or segments the channels; and fluid for regulating the microfluidic devices in the absence of external control. In some embodiments, the species are pressurized from at least one source with a pressure source selected from constant pressure, variable pressure, constant flow rate, or variable flow rate. In some embodiments, the segmentation is selected from the group consisting of a physical barrier, a chemical barrier, and an entropic barrier. In some embodiments, the species are solids, liquids, or gases. In some embodiments, the channels are voids in solid or semi-solid material. In some embodiments, the segmentation is coupled with an interfacing hole or holes to additional layers. In some embodiments, the segmentation comprises one or more valves, and wherein the device is capable of performing fluidic operations in the absence of external control. In some embodiments, the valves are two-way-valves, check-valves, capacitor-like valves or transistor-like-valves. In some embodiments, the system further comprises reagents for point of care applications (e.g., intravenous administration of fluids to a patient or intravenous administration of medication to a patient), reagents for diagnostic assays (e.g., immunoassays), reagents for research applications (e.g., drug screening assays, stem cell culture, protein function assays, or protein crystallization studies), or reagents for industrial applications. In some embodiments, the system further comprises a computer processor in contact with the devices, wherein the computer processor is configured to direct the operations of the devices. In some embodiments, the devices are configured to perform pulsatile fluidic operations. In some embodiments, the system is fully functional in the absence of electricity. In some embodiments, the channels are voids in elastomeric materials. In some embodiments, the segmentation is a physical barrier of elastomeric material. In some embodiments, the species are Newtonian fluids. In some embodiments, separated channels are joined by bypassing segmentation via elastic deformation into surroundings or void in substrate. In some embodiments, joined channels are separated via elastic deformation against the segmentation. In some embodiments, the pressure source is selected from compressed solid, liquid, gas, mechanically driven, or gravity driven.

Embodiments of the present invention further provide a method of performing microfluidic operations, comprising: contacting one or more microfluidics devices, wherein each of the microfluidic devices comprises two or more segmented species-containing channels, where the pressure of the species joins or segments the channel with a fluid for regulating the microfluidic devices in the absence of external control under conditions such that the device performs microfluidic operations using the fluids. In some embodiments, the species are pressurized from at least one source with a pressure source selected from constant pressure, variable pressure, constant flow rate, or variable flow rate. In some embodiments, the segmentation is selected from the group consisting of a physical barrier, a chemical barrier, and an entropic barrier. In some embodiments, the species are solids, liquids, or gases. In some embodiments, the channels are voids in solid or semi-solid material. In some embodiments, the segmentation is coupled with an interfacing hole or holes to additional layers. In some embodiments, the segmentation comprises one or more valves, and wherein the device is capable of performing fluidic operations in the absence of external control. In some embodiments, the valves are two-way-valves, check-valves, capacitor-like valves or transistor-like-valves. In some embodiments, the method performs an application including, but not limited to point of care applications (e.g., intravenous administration of fluids to a patient or intravenous administration of medication to a patient), diagnostic assays (e.g., immunoassays), research applications (e.g., drug screening assays, stem cell culture, protein function assays, or protein crystallization studies), or industrial applications. In some embodiments, a computer processor in contact with the devices directs the operations of the devices. In some embodiments, the devices are configured to perform pulsatile fluidic operations. In some embodiments, the method is performed in the absence of electricity. In some embodiments, the channels are voids in elastomeric materials. In some embodiments, the segmentation is a physical barrier of elastomeric material. In some embodiments, the species are Newtonian fluids. In some embodiments, separated channels are joined by bypassing segmentation via elastic deformation into surroundings or void in substrate. In some embodiments, joined channels are separated via elastic deformation against the segmentation. In some embodiments, the pressure source is selected from compressed solid, liquid, gas, mechanically driven, or gravity driven.

Additional embodiments are described herein.

### DESCRIPTION OF THE FIGURES

FIG. 1 shows interactive elastomeric components for self-controlled devices. (A) A three-layer composite of the check-valve and switch-valve are shown with their electronic counterparts, the diode and transistor, respectively. (B) Comparison between microfluidic oscillator and electronic oscillator, demonstrating that the two states of a microfluidic oscillator automatically produce an alternating output flow between two distinct solutions being simultaneously infused at a constant rate whereas the electronic circuit oscillator has no distinguishing output since there is no distinction between electrons.

FIG. 2 shows self-controlled fluid-circuits driving secondary fluid-circuits. (A) Fluid-circuit diagram for 1 state of a microfluidic oscillator providing input signals to a second fluid-circuit which distributes flow of two solutions to 4 outlets. (B) Oscillating pressure profile at solution inlets for an infusion rate of 10 ml/min which can be used as a clock-signal to drive other circuits (C) Actual image of the fluid-circuit in part A depicting the ability to control percentage of flow depending on the magnitude of the oscillator's input signal as it flows from the source to the outlet. (D) Graphs of solution composition for each outlet as a percentage of each solution for both states of the oscillator.

FIG. 3 shows automated fluid circuits for universal operations. (A) Fluid-circuit diagram of components integrated to sequentially switch between three solutions being infused simultaneously at a constant flow rate. (B) Actual images of four of the seven states with “X” and “O” representing closed and opened valves, respectively. (C) The same predefined sequential release shown in B can be applied to assays like ELISA, which require sequential alternation, deposition and washing between different reagents. (D) The same FSM mechanism enables most modern devices to work independently without user control and perform any desired task, by alternating between predefined states such as the colors in a traffic light, the minutes in a digital clock or timer, etc.

FIG. 4 shows large-scale integration of various components. (A) In early electronic devices, before the development of a method for large scale integration of components the number of components per device was low and fabrication was tedious. (B) A microfluidic circuit with 1010 integrated elastomeric components (17 check-valves and 993 switch-valves) fabricated on a single substrate. (C) Ability to interact with physical objects within solutions is an important capability for many applications, shown is a schematic of a filter membrane being incorporated into the three-layer PDMS device. (D) Image of 6 mm beads accumulating on a 1 mm pore-size filter membrane.

FIG. 5 shows the equivalent fluidic circuit of the switch valve developed in embodiments of the present invention.

FIG. 6 shows equivalent fluidic circuit model exploited to simulate the fluidic oscillator.

FIG. 7 shows a graph of both the simulated and experimental data for the oscillators switching frequency for various flow rates within its operating range. The inset is a simulated graph of the output flow profile for 1 μl/min flow rate.

FIG. 8 (A) shows an electronic diode with its geometric parameters that dictate its voltage parameters, similarly (but by a different mechansim), a microfluidic check-valve (also same for switch-valve) can have a pre-defined threshold pressure based on its geometry. Varying L1 changes the pressure-generated force acting on the membrane and has a nearly linear effect on the threshold pressure, whereas varying W changes both the force acting on the membrane and the force required to deflect the membrane leading to a non-linear effect on the threshold pressure as shown in B. FIG. 13C shows three check-valves of different widths in parallel being simultaneously pressurized by a multi-syringe pump at 10 μl/min.

FIG. 9 shows a scheme for cascading switching circuit in FIG. 8.

FIG. 10 shows a fabrication procedure for large-scale integration of components.

FIG. 11 shows a fluid-circuit diagram of a switching scheme based on bead accumulation using the embedded filters in FIG. 9.

FIG. 12 shows capacitor-valves powered by a single air-syringe pumping and mixing fluids.

### DEFINITIONS

To facilitate an understanding of the present invention, a number of terms and phrases are defined below:

The term “sample” in the present specification and claims is used in its broadest sense. On the one hand it is meant to include a specimen or culture. On the other hand, it is meant to include both biological and environmental samples. A sample may include a specimen of synthetic origin.

Biological samples may be animal, including human, fluid, solid (e.g., stool) or tissue, as well as liquid and solid food and feed products and ingredients such as dairy items, vegetables, meat and meat by-products, and waste. Biological samples may be obtained from all of the various families of domestic animals, as well as feral or wild animals, including, but not limited to, such animals as ungulates, bear, fish, lagamorphs, rodents, etc.

Environmental samples include environmental material such as surface matter, soil, water and industrial samples, as well as samples obtained from food and dairy processing instruments, apparatus, equipment, utensils, disposable and non-disposable items. These examples are not to be construed as limiting the sample types applicable to the present invention.

As used herein, the term “cell” refers to any eukaryotic or prokaryotic cell (e.g., bacterial cells such as E. coli, yeast cells, mammalian cells, avian cells, amphibian cells, plant cells, fish cells, and insect cells), whether located in vitro or in vivo.

As used herein, the term “cell culture” refers to any in vitro culture of cells. Included within this term are continuous cell lines (e.g., with an immortal phenotype), primary cell cultures, transformed cell lines, finite cell lines (e.g., non-transformed cells), and any other cell population maintained in vitro.

As used, the term “eukaryote” refers to organisms distinguishable from “prokaryotes.” It is intended that the term encompass all organisms with cells that exhibit the usual characteristics of eukaryotes, such as the presence of a true nucleus bounded by a nuclear membrane, within which lie the chromosomes, the presence of membrane-bound organelles, and other characteristics commonly observed in eukaryotic organisms. Thus, the term includes, but is not limited to such organisms as fungi, protozoa, and animals (e.g., humans).

As used herein, the term “in vitro” refers to an artificial environment and to processes or reactions that occur within an artificial environment. In vitro environments can consist of, but are not limited to, test tubes and cell culture. The term “in vivo” refers to the natural environment (e.g., an animal or a cell) and to processes or reaction that occur within a natural environment.

“Purified polypeptide” or “purified protein” or “purified nucleic acid” means a polypeptide or nucleic acid of interest or fragment thereof which is essentially free of, e.g., contains less than about 50%, preferably less than about 70%, and more preferably less than about 90%, cellular components with which the polypeptide or polynucleotide of interest is naturally associated.

The term “isolated” means that the material is removed from its original environment (e.g., the natural environment if it is naturally occurring). For example, a naturally-occurring polynucleotide or polypeptide present in a living animal is not isolated, but the same polynucleotide or DNA or polypeptide, which is separated from some or all of the coexisting materials in the natural system, is isolated. Such polynucleotide could be part of a vector and/or such polynucleotide or polypeptide could be part of a composition, and still be isolated in that the vector or composition is not part of its natural environment.

“Purified product” refers to a preparation of the product which has been isolated from the cellular constituents that the product is normally associated and from other types of cells which may be present in the sample of interest.

The terms “test compound” and “candidate compound” refer to any chemical entity, pharmaceutical, drug, and the like that is a candidate for use to treat or prevent a disease, illness, sickness, or disorder of bodily function. Test compounds comprise both known and potential therapeutic compounds. A test compound can be determined to be therapeutic by screening using the screening methods of the present invention. In some embodiments of the present invention, test compounds include antisense, siRNA or shRNA compounds.

As used herein, the term “processor” refers to a device that performs a set of steps according to a program (e.g., a digital computer). Processors, for example, include Central Processing Units (“CPUs”), electronic devices, or systems for receiving, transmitting, storing and/or manipulating data under programmed control.

As used herein, the term “memory device,” or “computer memory” refers to any data storage device that is readable by a computer, including, but not limited to, random access memory, hard disks, magnetic (floppy) disks, compact discs, DVDs, magnetic tape, flash memory, and the like.

### DETAILED DESCRIPTION OF THE INVENTION

The present invention relates to microfluidic devices. In particular, the present invention relates to microfluidic devices for performing spatio-temporal operations and applications thereof.

Unlike modern electronic systems where the controller and actuator circuits are all electrically driven, microfluidics currently requires peripheral electromechanical components for control and actuation of fluid flow (Pennathur, Lab Chip, 8, 383-387 (2008); Unger et al., Science 288, 113-116 (2000); Gu et al., Proc. Natl. Acad. Sci. USA 101, 15861-15866 (2004)). This more closely resembles the very early days of electrical circuitry where electromechanical relays performed electrical switching. There has been work in using two-phase flow interactions to regulate the movement of bubbles/droplets in order to perform logical operations which can direct the trailing pressurized fluid on-chip (Prakash et al., Science 315, 832-835 (2007); Cheow et al., Appl. Phys. Lett. 90, (2007)). This approach enables high-speed digital flow control, where the bubble/droplet represents a bit of information passing through logic gates, which can be useful for facilitating a multitude of chemical reactions requiring a set of sequential mixing steps. Although this approach can be very powerful for high-throughput droplet assays, this approach is not suitable for a significant portion of microfluidic research which deals with precisely regulating fluids to be exposed to or interact with other physical objects (i.e. microbeads (Lee et al., Science 16, 1793-1796 (2005), cells (Irimia et al., Lab Chip 6, 191-198 (2006), antibodies (Fan et al., Nature Biotech 26, 1373-1378 (2008)). In addition, the bubble/droplet approach requires dynamic input (dictating when bubbles/droplets should be created) in order to perform time-varying operations, which requires external controllers. Another approach, which aims to minimize the need for external control while providing on-chip regulation of fluid flow, is the use of embedded elastomeric valves with tuned resonant frequencies that respond passively according to the frequency of external inputs (Leslie D. C., et al. Frequency-specific flow control in microfluidic circuits with passive elastomeric features. Nature Physics 5, 231-235 (2009)). However, due to the large bandwidth of each component's resonant response, clean switching between different gates using different frequency external actuation is not achievable (Stone, Nature Physics 5, 178-179 (2009)). In addition, currently there is no scheme for different fluids to regulate each other in either a cascading or feedback mechanism.

Control in a cascading electrical or fluidic circuit is dictated by two parameters, a switching mechanism and a time delay. In the methods of embodiments of the present invention, the switching action is facilitated by check-valves and switch-valves that have geometrically defined threshold pressures. These components translate a constant infusion of fluid into a transient outflow. The steady infusion of fluid gradually pressurizes the compliant component until it discharges the pressure upon opening; this process mimics the time-delay effect of a charging capacitor.

Methods have been developed to overcome the non-linearity obstacle to perform fluidic-logic in a microfluidic platform. These systems are limited in applicability due to requirements for specialized polymer solutions, external electronic actuation to form bubbles, precise positioning of multiple droplets of different volumes onto a chip or by the limited non-linearity of the device response. Embodiments of the present invention provide a simple substrate architecture and scalable substrate processing methods that enables integration of multiple non-linearly responsive microfluidic components.

### I. Devices

In some embodiments, the present invention provides microfluidics devices for use in performing fluidic-logic, biochemical and industrial applications. The devices may be constructed of any suitable material. Exemplary, non-limiting examples of microfluidic devices are described below. In some embodiments, the devices comprise multiple segmented species-containing channels (e.g., valves), where the pressure of the species joins or segments the channels. In some embodiments, species (e.g., fluids or pressure) are used to regulate the opening or closing of the channels.

In some embodiments, devices are made by the sandwiching of three layers (e.g., poly-dimethylsiloxane (PDMS) layers). In some embodiments, the top and bottom layers contain the main network of microfluidic channels. The middle layer is a thin membrane.

In some embodiments, layers are made by supplying a negative “master” and casting a castable material over the master. Castable materials include, but are not limited to, polymers, including epoxy resins, curable polyurethane elastomers, polymer solutions (e.g., solutions of acrylate polymers in methylene chloride or other solvents), curable polyorganosiloxanes, and polyorganosiloxanes which predominately bear methyl groups (e.g., polydimethylsiloxanes (“PDMS”)). Curable PDMS polymers are well known and available from many sources. Both addition curable and condensation-curable systems are available, as also are peroxide-cured systems. All these PDMS polymers have a small proportion of reactive groups which react to form crosslinks and/or cause chain extension during cure. Both one part (RTV-1) and two part (RTV-2) systems are available. Additional curable systems are preferred when biological particle viability is needed.

In some embodiments, transparent devices are desirable. Such devices may be made of glass or transparent polymers. PDMS polymers are well suited for transparent devices. A benefit of employing a polymer which is slightly elastomeric is the case of removal from the mold and the potential for providing undercut channels, which is generally not possible with hard, rigid materials. Methods of fabrication of microfluidic devices by casting of silicone polymers are well known. See, e.g. D. C. Duffy et al., “Rapid Prototyping of Microfluidic Systems in Poly(dimethylsiloxane),” Analytical Chemistry 70, 4974-4984 (1998). See also, J. R. Anderson et al., Analytical Chemistry 72, 3158-64 (2000); and M. A. Unger et al., Science 288, 113-16 (2000), each of which is herein incorporated by reference in its entirety.

In some embodiments, fluids are supplied to the device by any suitable method. Fluids may, for example, be supplied from syringes, from microtubing attached to or bonded to the inlet channels, etc.

Fluid flow may be established by any suitable method. For example, external micropumps suitable for pumping small quantities of liquids are available. Micropumps may also be provided in the device itself, driven by thermal gradients, magnetic and/or electric fields, applied pressure, etc. All these devices are known to the skilled artisan. Integration of passively-driven pumping systems and microfluidic channels has been proposed by B. H. Weigl et al., Proceedings of MicroTAS 2000, Enshede, Netherlands, pp. 299-302 (2000).

In other embodiments, fluid flow is established by a gravity flow pump, by capillary action, or by combinations of these methods. A simple gravity flow pump consists of a fluid reservoir either external or internal to the device, which contains fluid at a higher level (with respect to gravity) than the respective device outlet. Such gravity pumps have the deficiency that the hydrostatic head, and hence the flow rate, varies as the height of liquid in the reservoir drops. For many devices, a relatively constant and non-pulsing flow is desired.

To obtain constant flow, a gravity-driven pump as disclosed in published PCT application No. WO 03/008102 A1 (Jan. 18, 2002), herein incorporated by reference, may be used. In such devices, a horizontal reservoir is used in which the fluid moves horizontally, being prevented from collapsing vertically in the reservoir by surface tension and capillary forces between the liquid and reservoir walls. Since the height of liquid remains constant, there is no variation in the hydrostatic head.

Flow may also be induced by capillary action. In such a case, fluid in the respective outlet channel or reservoir will exhibit greater capillary forces with respect to its channel or reservoir walls as compared to the capillary forces in the associated device. This difference in capillary force may be brought about by several methods. For example, the walls of the outlet and inlet channels or reservoirs may have differing hydrophobicity or hydrophilicity. Alternatively, the cross-sectional area of the outlet channel or reservoir is made smaller, thus exhibiting greater capillary force.

In some embodiments, flow is facilitated by embedded capacitor valves that pump fluids in a separate channel when pressurized. This is achieved by having a series of valves in the bottom that direct a pressurized gas or liquid causing the membrane to deform and squeeze the fluid in the top channel forward. Additional control is provided by having valves in the top layer that can open sequentially.

### II. Uses

In some embodiments, a fluidic system comprising valves and channels is built to perform a particular task. Usually this means a certain number of valves need to be operated, for example, opened or closed, in a particular sequence, and possibly for different durations, in order to accomplish the desired task.

One advantage of the systems and methods of embodiments of the present invention is the auto-regulatory role of the components and the ability to incorporate them in large scale both in parallel and in series. This advantage allows complex operations to be performed with little external setup or signaling. For cases where a pre-defined operation is desired without any variable decisions, this system enables all functionality to be completely encoded into the device (that is no external electrical, pneumatic, or mechanical input is required except for the source of fluid flow). This allows users with little training to operate the devices. Therefore, in some embodiments, the systems and methods described herein are amenable for point-of-care applications, diagnostic tests, and assays for non-microfluidic specialists in academic or industrial labs. For assays which require a specific sequence and ratio of mixing of solutions (e.g., immunoassays/drug screenings/crystallization studies), the systems described herein can perform the assays automatically with the user only needing to activate the device.

However, some assays require a variable input from the user to which the device then subsequently performs a particular operation. In this case, it is preferred to have some kind of logical control over the device's operations. The systems described herein enable logical operations to be performed by reconfiguring the geometry through input signals from the user that can either open or close transistor-valves. An example would be the metering of solutions based on a patient's weight. The device can have several inputs for different weight ranges which respectively activate a separate set of components which will deliver different amounts of solutions.

Many cells and biological systems respond to the same chemical or set of chemicals differently depending on spatio-temporal pattern of administration of the drug or biochemical.

For example, insulin is released in a pulsatile manner in the body; some bacteria grow better when provided with nutrients in waves rather than constantly or in a onetime bolus; G-protein coupled receptor (GPCR) signaling can be very different depending on the temporal pattern of ligand delivery to cells. This capability can be used to determine signaling mechanisms as well as potentially regulate cell behavior. Thus the ability of the embedded fluidic circuits to provide various pulsed patterns of chemicals to cells has important applications in stem cell proliferation and differentiation, in determining signaling mechanisms, and in optimizing bioreactor production, etc. The small size and multiplexing capabilities especially are useful for screening many different temporal patterns of chemical exposure on cells in parallel to identify ideal conditions for culture, differentiation, mechanistic evaluations.

Another advantage of the systems and methods of embodiments of the present invention are their ability to compartmentalize fluids in an unpressurized state, providing a means for device memory. Therefore assays can be conducted which provide several output solutions which can be stored and segregated from other fluids by negating any flow or diffusion of molecules which could cross-contaminate the solutions. For example, in some embodiments, a sample of blood that contains unstable proteins is taken from a patient. Those proteins are then be immediately processed by the device and provide a preliminary diagnosis while also being stored in a stable solution so that testing can be performed later in a lab if needed. This feature is particularly useful for point-of-care applications where there could be a long distance from the ground site and the testing lab which is expensive and inconvenient to have patients unnecessarily travel.

The devices, system and method of the present invention find use in a variety of applications including, but not limited to, point of care diagnostics (e.g., field assays and tests, especially where electrical power sources are not available or where electrical circuits might not be durable (e.g., in space or nuclear power plants)); mechanistic studies, bioreactor optimization, stem cell culture, and crystallization studies.

Additional applications of microfluidic devices include, but are not limited to, chemical and biochemical sensing systems, protein and chemical synthesis, and spectroscopy of ultra-small volumes. Depending on the application, the microfluidic devices of the invention can provide as output fluidic logic signals, processed materials (e.g., such as micro- or nano-quantities of chemicals or other substances), or information about the materials that are processed, such as the results of diagnostic tests, that can be of significance for a user.

### EXPERIMENTAL

The following examples are provided in order to demonstrate and further illustrate certain preferred embodiments and aspects of the present invention and are not to be construed as limiting the scope thereof.

### Example 1

### A. Methods

### Methods Summary

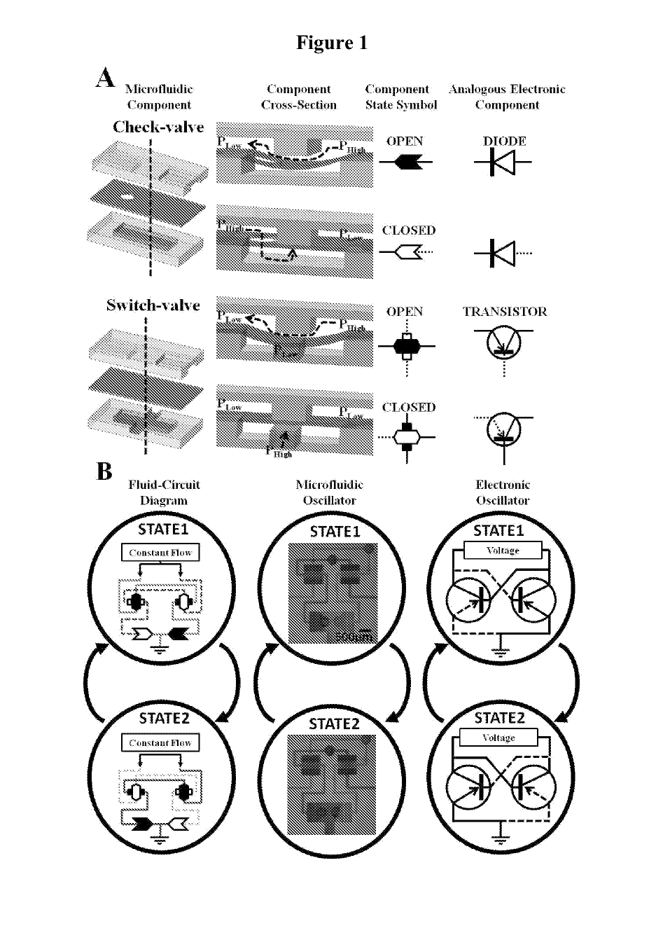

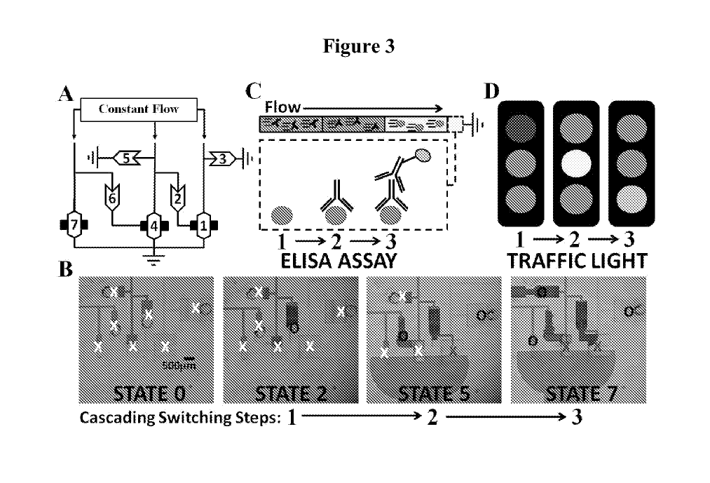

The device consists of three layers made from PDMS prepolymer and curing agent (Sylgard 184, Dow Corning Co., Midland, Mich.) at a 10:1 ratio. The top and bottom layers are molded against a master mold made by standard photolithography using the negative-photoresist SU-8 (SU-8, MicroChem. Co., Newton, Mass.). The master molds were silanized in a desiccator for 2 hours (United Chemical Tech., Bristol, Pa.). The height of all top layer and bottom layer features is 100 μm except for those in FIG. 1 which are 50 μm and the top layer of FIG. 3 which is 30 μm. The PDMS molds of the top and bottom layers were cured in a 60° C. oven for over 2 hours. The middle-layer membrane was made by spin-coating a 30 μm PDMS layer on a silanized silicon-wafer at 1500 rpm for 90 seconds and then curing in a 120° C. oven for 30 min. All layers were bonded together using oxygen plasma treatment (SPI Plasma-Prep II, Structure Probe, Inc., West Chester, Pa.) for 30 seconds. The filter used was a 2 mm diameter punch out of the 1 μm Cyclopore track-etched polycarbonate thin clear membrane (Whatman, Piscataway, N.J.). The 6.0 μm microbeads were made of polystyrene (Polysciences Inc., Warrington, Pa.).

Fabrication of Device with Integrated Components

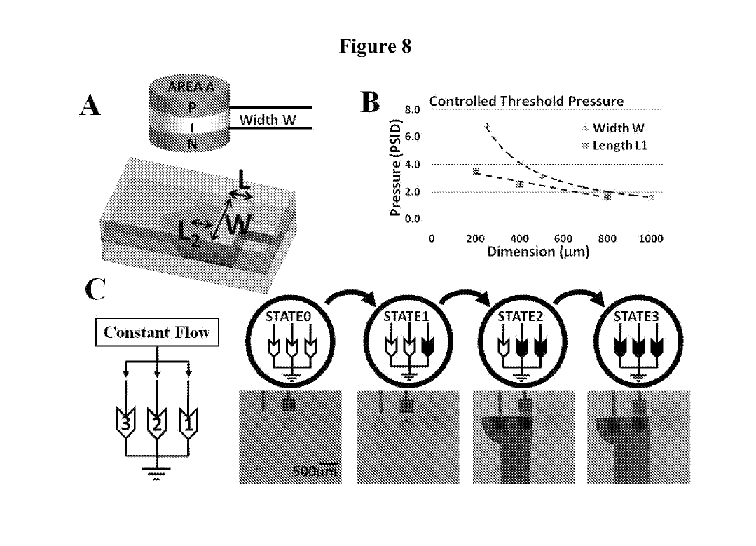

The three layers of the device are bonded. In the first step, the thin PDMS membrane, while still on the silicon wafer (or any flat substrate), is bonded to either the top or bottom layer and then the two bonded layers are detached from the wafer. Access holes are made in the top layer with a biopsy punch. Holes are punched into the bonded middle layer, using a 350 μm biopsy punch (Ted Pella Inc., Redding, Calif.), for the check-valve and switch-valve components for all figures except FIG. 8E which used a 29 gauge insulin syringe. For check-valve components, a hole is punched in the downstream region of the cavity directly after the gap. Switch-valves have holes punched to interface access channels of the top and bottom layers. To ensure efficacy of the components, the circular shape of the biopsy punch can be molded into either or both the bottom layer and top layer so the hole is accurately punched every time (as shown in FIG. 8A). For the second step, the top layer and the bonded middle and bottom layers are exposed to oxygen plasma for 30 seconds. In the third step, the gap regions of the components are rendered unable to permanently bond to the middle membrane by stamping those surfaces with a non-oxidized PDMS stamp. The PDMS stamp was made by fabricating the negative of the cavity regions on the master mold so that those regions extrude out. By this method, when many components are integrated on a single device, a template with exact spacing is made so that only one stamp is needed to selectively pattern the binding between the PDMS layers. Finally in step four, all layers were aligned and bonded together and the device was incubated at 60° C. for 1 minute to enhance the bond strength. Solutions were then inserted in the access holes to fill the device. The device is not used directly after being placed in a vacuum since it can cause some components to open; in which case waiting about 30 minutes allows air to penetrate back in. Tubing or other interfaces can be connected to access holes in the top layer.

### Component Pressure Characterization

The dependence of threshold pressure on the geometry of a component was characterized for three lengths of both L1 and W (as shown in FIG. 8B). The threshold pressure was determined by continuously measuring the differential pressure across the component with a differential pressure transducer (Model PX139-005D4V, Omega Eng. Inc., Stamford, Conn.) as a syringe pump continuously pressurized the microchannel with a flow-rate of 6 ml/hr. The pressure transducers were connected to access microchannels that were located directly before and after the component for quicker response timing and more accurate readings. The threshold pressure for a given trial was determined by the peak pressure in the pressure histogram as measured by the transducer as the component pressurized to a critical limit and opened. The average of three trials was plotted to give the relationship between the threshold pressure and the component dimension. The graph of FIG. 3B shows the different threshold pressures for both a constant W of 1 mm with L1 values of 800 μm, 400 μm, and 200 μm as well as for a constant L1 of 800 μm with W values of 1000 μm, 500 μm, and 250 μm. For all components, L2 was maintained constant at 300 μm. The oscillator pressure measurements were performed by measuring the gauge pressure of both inlets simultaneously using two pressure transducers (Model PX26-015DV, Omega Eng. Inc., Stamford, Conn.). Measurements were taken every 100 ms and the graphs are the moving time-average of 50 minus the measured zero value for each sensor.

### B. Results

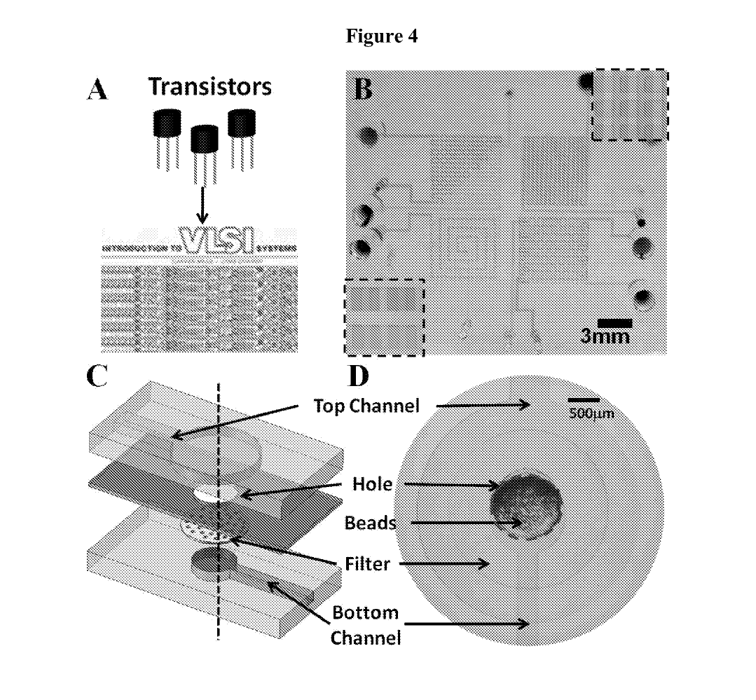

All components in the fluidic circuits are made in a three layer polydimethylsiloxane (PDMS) substrate where the top and bottom layers contain patterned channel features and cavities whereas the middle layer is a thin deformable membrane with strategically positioned through-holes (FIG. 1A). Both the check-valve and switch-valve consist of an interrupted microchannel in one layer, a cavity in the other layer, and a deformable membrane in between that can deflect into the cavity to allow the interrupted channel to become connected. The check-valve also has a through-hole in the membrane layer to connect one of the ends of the interrupted microchannel with the cavity on the opposing layer. The position of this through-hole dictates the direction of flow allowed and effectively creates a diode-like function which negates any back-flow and diffusion in its closed state (see below). The check-valve can withstand back-pressures (with zero back-flow) up to 45 psi, which is the bonding strength of PDMS (Eddings et al., J. Micromech. and Microeng. 18, 067001 (2008)); this improves on previous passive monolithic check-valve designs by more than 15 psi (Jeon et al., Biomed. Microdev. 4, 117-121 (2002); Adams et al., J. Micromech. and Microeng 15, 1517-1521 (2005); Yang et al., Sensors and Actuators A 134, 186-193 (2007); Loverich et al., Microfluid. Nanofluid. 3, 427-435 (2007)). A switch-valve is flow-permissive in both directions but can have access channels to its cavity so that an alternate pressure can force the switch-valve into a closed “off” state. A switch-valve with two access channels is shown (FIG. 1A); alternatively it can also have either zero or one access channels as can be seen in the bottom left inset of FIG. 4B and FIG. 3B, respectively.

Integrating components in specific configurations enables pre-defined regulation of fluids. A fundamental fluidic operation, which has only been able to be facilitated by external control, is the continuous switching of single-phase Newtonian fluids. FIG. 1B shows the two states of a fluid-circuit diagram of a microfluidic oscillator which is comprised of two switch-valves each connected to a check-valve. These interactive components enable two constant input flows to self-regulate each other to indefinitely oscillate their output flow in an alternating fashion so that only one fluid is flowing at a given time, as seen by the switching of the output flow in FIG. 1B. Here, when the first fluid reaches a threshold pressure, it breaks through and opens one switch-valve (right side) and flows through to the cavity chamber of the other switch-valve (left side) to close and turn that off. Subsequently, a check valve opens to let the fluid flow to the outlet and release the pressure. Now the second fluid builds up pressure while the pressure of the first fluid decreases to repeat the process on the other side of the circuit. The check-valves serve two purposes; they provide needed resistance to maintain enough pressure in the cavity chamber to turn off the switch-valve before the fluid flows out to the outlet. Secondly, the check-valves ensure that there is no back-flow, eliminating cross-contamination of the fluid species as they switch. There is a linear relationship between flow rate and switching frequencies (see below) for a range of flow rates. In this linear range, the increase in frequency with increasing flow reflects decrease in time for threshold pressure to build up (time for parameters C₁/I₁, C₂/I₂ to surpass a threshold pressure in the model). However, at flow rates higher than this operating range, the switching frequencies approach the response time of the valve opening and closings resulting in partially switching oscillations. Eventually, switching frequencies surpass valve response times substantially resulting in steady adjacent laminar flow of the two solutions. The operating range which enables a full switching oscillation to occur is determined by the resistance provided by the components and geometries that dictate timing of the pressurizing and depressurizing of the fluids in the cavity chamber as well as the response time of the membrane's elasticity. The good agreement between experiments and prediction from a simple computer model also supports the usefulness of computer assisted design of circuits.

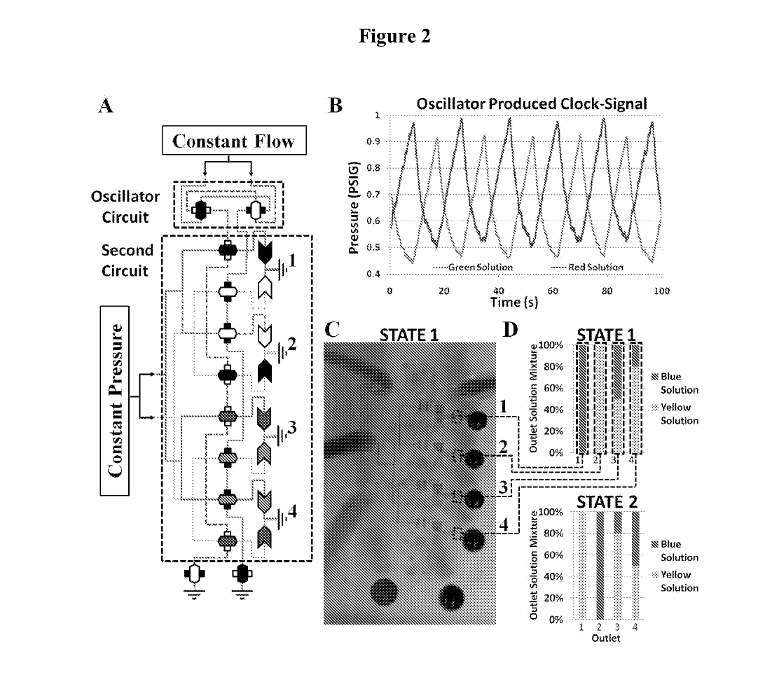

Creating oscillations on-chip has many implications to signal processing, clock-signal generation, and also biological relevance for applications which utilize cyclic or pulsatile flow. FIG. 2 demonstrates how the microfluidic oscillator can be used to control other fluid-circuits that regulate flow of different solutions. FIG. 2A is a fluid-circuit diagram that uses inputs (first and second solutions) from the oscillator (previously shown in FIG. 1B) to control the switching between the states of a second fluid-circuit. The oscillation in pressure of the solutions (at the inlet of each solution) is shown in FIG. 2B which is used as a clocking signal and controller for the second fluid-circuit. The second fluid-circuit is comprised of eight switch-valves and eight check-valves that distribute flow of two sample solutions, powered by a constant pressure, to four outlets. The oscillator is connected to the second fluid-circuit such that the flow from each output state (either the first or second solution) activates the switch-valves of alternate sample solutions to each outlet at a single time. It can be seen that for state 1, when outlet 1 is a first colour, outlet 2 is a second colour (FIG. 2C); alternatively for state 2 when outlet 1 is the first colour, outlet 2 is the second colour. Outlets 3 and 4 show how partial closure of valves can be utilized in order to achieve mixtures of solutions by taking the input signal at a lower pressure region. FIG. 2D shows the distribution of flow to each outlet for each of the two states of the oscillator.

In addition to oscillations, another useful fluidic control function is the ability to perform an automated sequential operation as done in electronic finite state machines. FIG. 8 shows how channel networks can be automatically reconfigured which enables different fluidic operations to be performed sequentially. This is achieved by having components of different threshold pressures, dictated by their physical geometry, so that they are activated at different times when being infused simultaneously. These threshold pressures combined with capacitance of the elastomeric channels and components enable a time-regulated discretization of flow conduction into on and off states. Cascading processes are achieved using interacting networks of components with pre-defined threshold pressures both in parallel and in series. FIG. 3A is a fluid-circuit diagram that automatically switches between three solutions using the systematic activation of seven components, four check-valves and three switch-valves. The sequence at which each component changes state is designated in the fluid-circuit diagram (see below for circuit diagram for all seven states). The three solutions are simultaneously infused by a multi-syringe pump, or alternatively by the squeezing force of a clamp that simultaneously pressurizes three fluid reservoirs. Actual images of four of the seven states are shown in FIG. 3B, an “O” or “X” represents the component in an on or off state, respectively. An “X” designates a check-valve with a downstream channel linked to the cavity of a switch-valve. This combination of components locks in pressure which maintains both components in an off state despite any subsequent release of pressure behind the check-valve; this demonstrates the capability for on-chip memory (See below for a description of the scheme for the cascading switching mechanism).

As with electronic circuits, one factor for wide-spread use of microfluidic devices is the ability to integrate components in large-scale (FIG. 4A). An efficient method to ensure that the middle membrane layer does not bind to the valve's gap region was developed by exploiting the known contamination of residual PDMS monomer from the surface of a non-oxidized PDMS stamp used for micro-contact printing (Yang et al., Langmuir 16, 7482-7492 (2000)). The effectiveness of this method was demonstrated by integrating over a 1000 components within a single substrate (FIG. 4B). Another aspect of microfluidic control that is enabled with embedded valves is self-regulation based on interaction with physical samples. As a demonstration it was shown that a single fluid can be subsequently released to different outlet channels based on the accumulation of microbeads on a filter membrane. FIG. 4C is a schematic of a semi-porous filter membrane being sandwiched within the three-layer PDMS device. The filter membrane is kept in place by the exerted pressure of the fluid on the elastic PDMS membrane as flow occurs through the punched hole to the bottom channel. The filter serves to block beads in the solution so that as they accumulate (FIG. 4D), the pressure in the device rises and eventually opens a subsequent valve as shown in FIG. 4C. In addition to bead accumulation, this sample-response mechanism is utilized with other physical objects such as cells or a precipitant from a chemical reaction. By designing larger fluid-circuits, more sophisticated operations including sequential switching steps of different solutions can be integrated to perform auto-adjusting sample preparation procedures on-chip where the timing constants are regulated depending on the sample properties such as particle concentration.

### Model

In order to estimate the performance of the developed microfluidic system, a theoretical model based on equivalent fluidic circuit concept was constructed. The underlying fluid model is based on the Navier-Stokes equation and mechanics. There are three basic components: fluid resistance, capacitance, and inductance that are used to derive the model.

### A. Fluid Resistance

Analogous to electrical resistance, fluid resistance is defined as the ratio of pressure drop over flow rate,

$$R = \frac{Δ P}{Q} in \frac{N \cdot s}{{m}^{5}}$$

where ΔP is the pressure difference, in N/m², and Q is the volume flow rate, in m³/s. For a microfluidic channel with a rectangular cross section with width w, and depth h, and assuming both-laminar flow and Newtonian fluid, the resistance is

$$R = {\frac{12 μ L}{w \cdot {h}^{3}} [ 1 - \frac{h}{w} ( \frac{192}{{π}^{5}} {\sum }_{n = 1}^{∞} \frac{1}{{n}^{5}} tanh ( \frac{n π w}{h} ) ) ]}^{- 1}$$

### B. Fluid Capacitance

Compliant elements of a fluidic system exhibit the fluidic equivalent of capacitance as a pressure-dependent volume change

$$C = \frac{d V}{d P} in \frac{{m}^{5}}{N}$$

The fluidic capacitance for a square membrane can be derived by plate theory as

$$C = \frac{6 {w}^{6} ( 1 - {v}^{2} )}{{π}^{4} {Et}^{3}}$$

where w is membrane width, in m, E is Young's modulus of membrane, in N/m², t is membrane thickness, in m, and v is Poisson's ratio of membrane (dimensionless.)

### C. Fluidic Inductance

In a manner analogous to electrical inductance, fluidic systems are capable of storing kinetic energy in fluidic inductance, H (in kg/m⁴)

$$Δ P = H \frac{d Q}{d t}$$

For incompressible and inert fluids in tubes of constant cross section A, the fluidic inductance is given by

$$H = \frac{ρ L}{A}$$

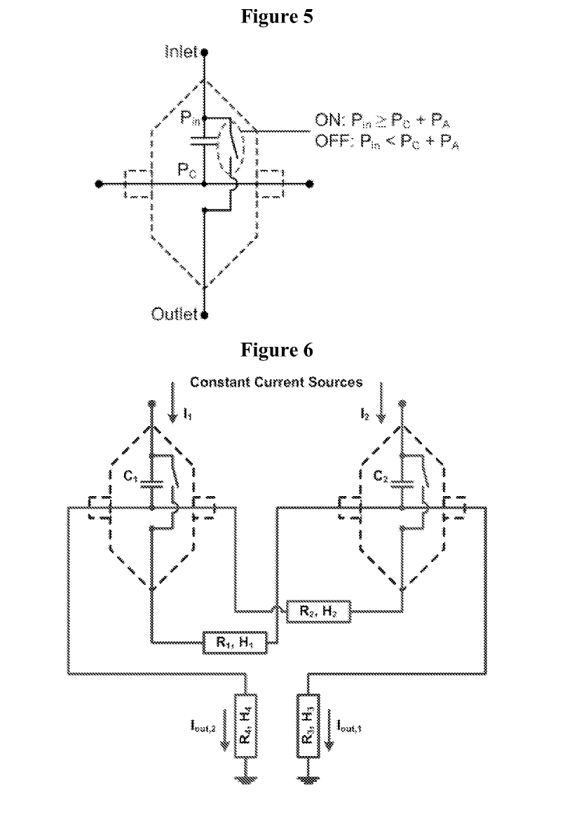

The switch valve was modelled it as a capacitor between the inlet and the cavity channel, and a switch between the inlet and the outlet (FIG. 5). While the inlet pressure (Pᵢₙ) is lower than the summation of cavity pressure (P_{C}) and a pressure due to the adhesion between the PDMS surfaces (P_{A}), the valve (switch) is closed. Therefore, the inlet pressure will be built up like a capacitor due to the compliance of the membrane. Once the inlet pressure reaches the threshold, the membrane is deformed down allowing the liquid to flow from the inlet to the outlet. As a result, the valve (switch) is turned on to discharge the flow from the inlet, which can be analogous to discharging a capacitor. Using the aforementioned equivalent circuit components, the equivalent fluidic circuit was exploited as shown in FIG. 10 with the values listed in Table 1 (calculated using above equations) to model the behaviour of the oscillator fluidic network.

TABLE 1

| Component | Value |
|---|---|
| C₁, C₂ | 5.78 × 10⁻¹⁵ (m⁵/N) |
| Rₗ | 1.21 × 10¹² (N?s/m⁵) |
| H₁ | 3.50 × 10⁸ (kg/m⁴) |
| R₂ | 1.56 × 10¹² (N?s/m⁵) |
| H₂ | 4.50 × 10⁸ (kg/m⁴) |
| R₃, R₄ | 1.18 × 10¹² (N?s/m⁵) |
| H₃, H₄ | 3.40 × 10⁸ (kg/m⁴) |

### Additional Analysis

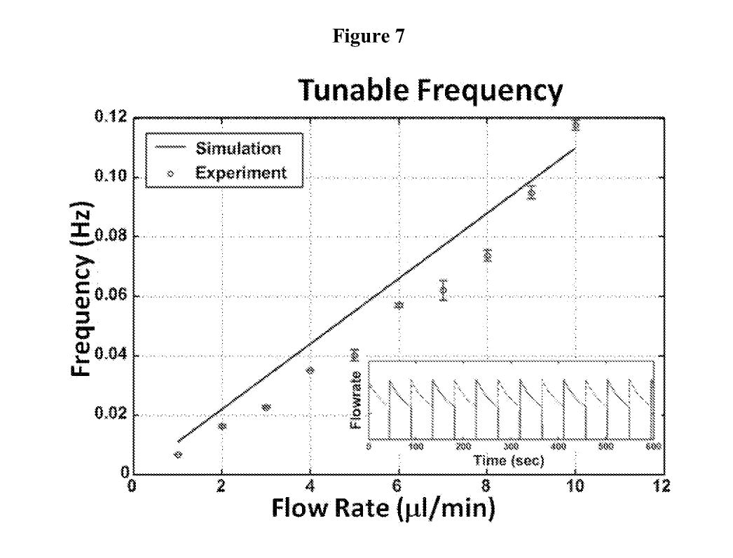

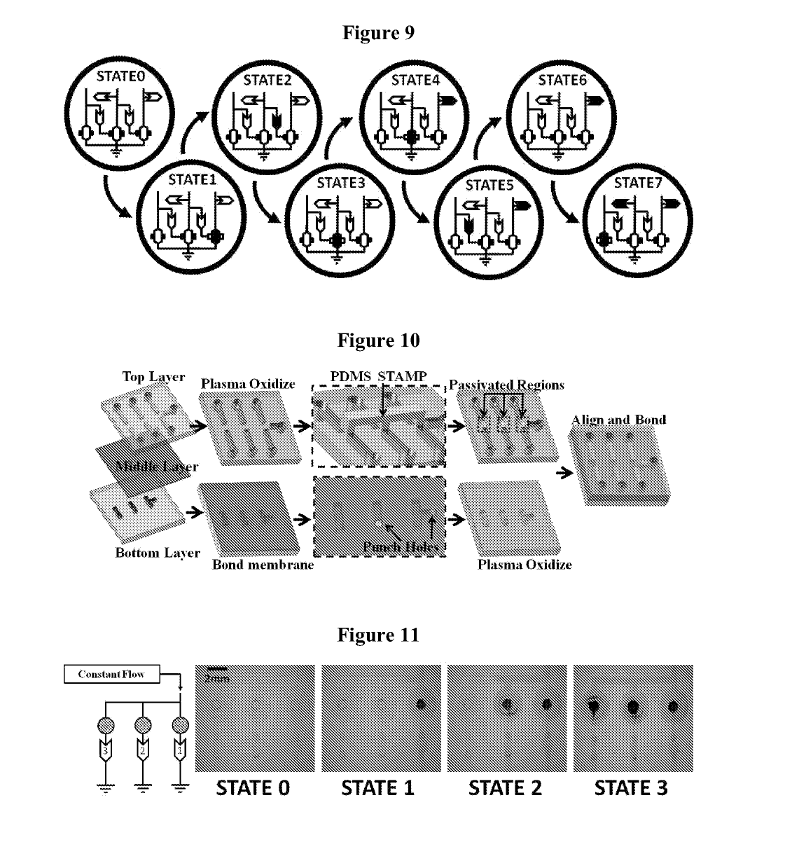

FIG. 7 shows a graph of both the simulated and experimental data for the oscillators switching frequency for various flow rates within its operating range. FIG. 8 shows an electronic diode with its geometric parameters that dictate its voltage parameters, similarly (but by a different mechansim), a microfluidic check-valve (also same for switch-valve) can have a pre-defined threshold pressure based on its geometry. W and L1 are the width and length, respectively, of the overlap region between the upper layer channel and lower layer cavity. L2 is the length of the gap region between the two parts of the interrupted channel. To open the valve, the differential pressure of the fluid needs to overcome two forces: the adhesive force between the PDMS layers and the elastic force arising from the deformation of the middle layer. Varying L1 changes the pressure-generated force acting on the membrane and has a nearly linear effect on the threshold pressure, whereas varying W changes both the force acting on the membrane and the force required to deflect the membrane leading to a non-linear effect on the threshold pressure as shown in FIG. 8. This geometry-based design principle allows pre-defined operating threshold pressures to be set for the check-valve as well as for the switch-valve in a predictable manner. FIG. 9 shows three check-valves of different widths in parallel being simultaneously pressurized by a multi-syringe pump at 10 μl/min. The check-valves open sequentially in order from the largest width to the smallest width.

FIG. 10 shows a scheme for Cascading Switching Circuit in FIG. 8. The precise activation sequence was facilitated by varying threshold pressures through changing W for components of the three different fluids and both L₁ and L₂ for components of the same fluid. In this scheme, after the first switch-valve containing a first solution is opened, the pressure from the adjacent second fluid opens a check-valve that is connected to the cavity of the open first fluid switch-valve, causing the switch-valve to turn off. As the first fluid pressure continues to build due to the now closed switch-valve, a subsequent venting check-valve opens to release the pressure of the first fluid. The process repeats itself when the switch-valve opens and releases the second fluid. These functions are microfluidic analogues to the IF-ELSE functions of transistors in electrical circuits.

Since all fluidic components are made within the same three layers, their integration into a single device is highly scalable. The fabrication procedure which selectively deactivates oxidized PDMS layers so that the middle membrane layer does not bond to the part of the PDMS that forms the gap between interrupted channels is useful for such integration (FIG. 11).

FIG. 11 shows a sample-based switching mechanism based on bead accumulation using the embedded filters in FIG. 4. Silver circles represent embedded filter membranes and numbers in components represent order of actuation dictated by varying check-valve width.

In summary, this example describes a substrate-architecture, circuit-design principles, and a scalable fabrication process to construct interactive microfluidic flow-controlling component networks. Simple variation of component geometry directly controls its opening threshold pressure enabling control of timing of flow valving and switching. Although the fluidic control demonstrations shown here are simple, electronic circuit analysis describes that every circuit or logic operation is possible using only a transistor component. Since a transistor's directionally distinct switching properties are mimicked by having a switch-valve and check-valve in series, this demonstrates broad applicability of the developed elastomeric components for device embedded flow control.

### Example 2

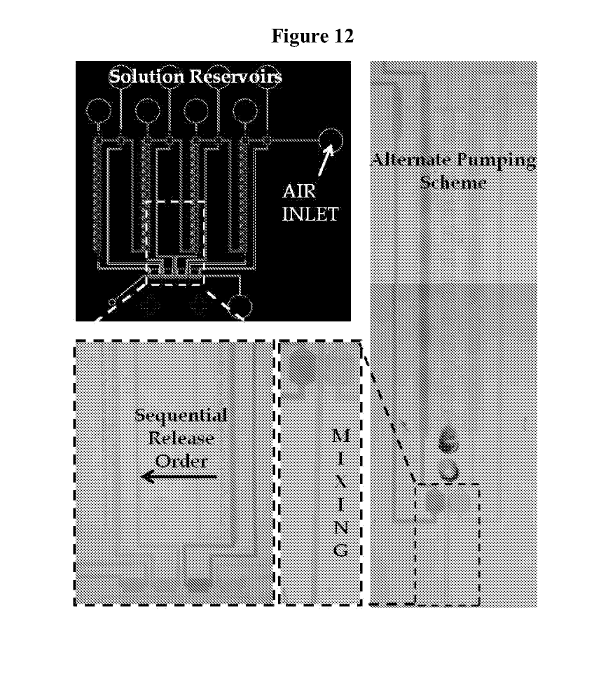

This Example describes the use of an air-driven device. The capacitance of an elastomer is used to pump fluids in a separate channel when pressurized. This is achieved by having a series of valves in the bottom that direct a pressurized gas or liquid causing the membrane to deform and squeeze the fluid in the top channel forward. Additional control is provided by having valves in the top layer that can open sequentially as previously demonstrated. FIG. 12 shows a scheme of using a series of components to direct a pneumatic force in a peristaltic fashion to pump and mix a multitude of fluids in specific orders powered by a single air-filled syringe. This system is used to perform complex assays (e.g., for point-of-care applications).

All publications and patents mentioned in the above specification are herein incorporated by reference. Various modifications and variations of the described method and system of the invention will be apparent to those skilled in the art without departing from the scope and spirit of the invention. Although the invention has been described in connection with specific preferred embodiments, it should be understood that the invention as claimed should not be unduly limited to such specific embodiments. Indeed, various modifications of the described modes for carrying out the invention which are obvious to those skilled in electrical engineering, optics, physics, and molecular biology or related fields are intended to be within the scope of the following claims.

1. A system, comprising:
a) one or more microfluidics devices, wherein each of said microfluidic devices comprises two or more segmented species-containing channels, where the pressure of said species joins or segments said channels; and
b) fluid for regulating said microfluidic devices in the absence of external control.

2. The system of claim 1, wherein said species are pressurized from at least one source with a pressure source selected from the group consisting of constant pressure, variable pressure, constant flow rate, and variable flow rate.

3. The system of claim 1, wherein said segmentation is selected from the group consisting of a physical barrier, a chemical barrier, and an entropic barrier.

4. The system of claim 1, wherein said species are selected from the group consisting of solids, liquids, and gases.

5. The system of claim 1, wherein said channels are voids in solid or semi-solid material.

6. The system of claim 1, wherein said segmentation is coupled with an interfacing hole or holes to additional layers.

7. The system of claim 1, wherein said segmentation comprises one or more valves, and wherein said device is capable of performing fluidic operations in the absence of external control.

8. The system of claim 7, wherein said valves are selected from the group consisting of two-way-valves, check-valves, capacitor-like valves and transistor-like-valves.

9. The system of claim 1, further comprising reagents selected from the group consisting of reagents for point of care applications, reagents for diagnostic assays, reagents for research applications, and reagents for industrial applications.

10. The system of claim 9, wherein said point of care operations are selected from the group consisting of intravenous administration of fluids to a patient and intravenous administration of medication to a patient.

11. The system of claim 9, wherein said research applications are selected from the group consisting of drug screening assays, stem cell culture, protein function assays, and protein crystallization studies.

12. The system of claim 9, wherein said diagnostic assays are immunoassays.

13. The system of claim 1, further comprising a computer processor in contact with said devices, wherein said computer processor is configured to direct the operations of said devices.

14. The system of claim 1, wherein said devices are configured to perform pulsatile fluidic operations.

15. The system of claim 1, wherein said system is fully functional in the absence of electricity.

16. The system of claim 1, wherein said channels are voids in elastomeric materials.

17. The system of claim 1, wherein said segmentation is a physical barrier of elastomeric material.

18. The system of claim 1, wherein said species are Newtonian fluids.

19. The system of claim 1, wherein separated channels are joined by bypassing segmentation via elastic deformation into surroundings or void in substrate.

20. The system of claim 19, wherein joined channels are separated via elastic deformation against said segmentation.

21. The system of claim 2, where said pressure source is selected from the group consisting of compressed solid, liquid, gas, mechanically driven, and gravity driven.

22. A method of performing microfluidic operations, comprising:
contacting one or more microfluidics devices, wherein each of said microfluidic devices comprises two or more segmented species-containing channels, where the pressure of said species joins or segments said channel with a fluid for regulating said microfluidic devices in the absence of external control under conditions such that said device performs microfluidic operations using said fluids.
